(19) United States
(12) Patent Application Publication
(10) Pub. No.: US 2011/0124652 A1
(43) Pub. Date: May 26, 2011

(54) Chemical Compounds and Uses

(76) Inventors: Andrew J. Carpenter, Durham, NC (US); Jing Fang, Durham, NC (US); Gregory Peckham, Durham, NC (US)

(21) Appl. No.: 13/056,339

(22) PCT Filed: Jul. 28, 2009
(86) PCT No.: PCT/US09/51939

§ 371 Date: Jan. 28, 2011

(30) Foreign Application Priority Data
Jul. 30, 2008 (US) 61084754

(51) Int. Cl. A61K 31/501; C07D 401/14; A61P 3/10; A61P 3/04

(52) U.S. Cl. 514/252.02; 544/238

(57) ABSTRACT

The present invention relates to novel compounds that are useful in the treatment of metabolic disorders, particularly type II diabetes mellitus and related disorders, and also to the methods for the making and use of such compounds.

### FIELD OF THE INVENTION

The present invention relates to novel compounds that are useful in the treatment and prevention of metabolic disorders, including diabetes mellitus (type I and type II), obesity, and related disorders, and also includes methods for making, pharmaceutical compositions containing, and therapeutic uses for such compounds.

### BACKGROUND OF THE INVENTION

Diabetes mellitus is an ever-increasing threat to human health. For example, in the United States current estimates maintain that about 16 million people suffer from diabetes mellitus.

Type I diabetes, also known as insulin-dependent diabetes mellitus (IDDM), is caused by the autoimmune destruction of the insulin producing pancreatic 13-cells, and necessitates regular administration of exogenous insulin. Without insulin, cells cannot absorb sugar (glucose), which they need to produce energy. Symptoms of Type I diabetes usually start in childhood or young adulthood. People often seek medical help because they are seriously ill from sudden symptoms of high blood sugar (hyperglycemia).

Type II diabetes, also known as non-insulin-dependent diabetes mellitus (NIDDM), manifests with an inability to adequately regulate blood-glucose levels. Type II diabetes may be characterized by a defect in insulin secretion or by insulin resistance, namely those that suffer from Type II diabetes have too little insulin or cannot use insulin effectively. Insulin resistance refers to the inability of body tissues to respond properly to endogenous insulin. Insulin resistance develops because of multiple factors, including genetics, obesity, increasing age, and having high blood sugar over long periods of time. Type II diabetes, sometimes called mature or adult onset diabetes, can develop at any age, but most commonly becomes apparent during adulthood. The incidence of Type II diabetes in children, however, is rising.

In diabetics, glucose levels build up in the blood and urine causing excessive urination, thirst, hunger, and problems with fat and protein metabolism. If left untreated, diabetes mellitus may cause life-threatening complications, including blindness, kidney failure, and heart disease.

Type II diabetes accounts for approximately 90-95% of diabetes cases, killing about 193,000 U.S. residents each year. Type II diabetes is the seventh leading cause of all deaths. In Western societies, Type II diabetes currently affects 6% of the adult population with world-wide frequency expected to grow by 6% per annum. Although there are certain inheritable traits that may predispose particular individuals to developing Type II diabetes, the driving force behind the current increase in incidence of the disease is the increased sedentary lifestyle, diet, and obesity now prevalent in developed countries. About 80% of diabetics with Type II diabetes are significantly overweight. As noted above, an increasing number of young people are developing the disease. Type II diabetes is now internationally recognized as one of the major threats to human health in the 21^{st} century.

Type II diabetes currently is treated at several levels. A first level of therapy is through the use of diet and/or exercise, either alone or in combination with therapeutic agents. Such agents may include insulin or pharmaceuticals that lower blood glucose levels. About 49% of individuals with Type II diabetes require oral medication(s), about 40% of individuals require insulin injections or a combination of insulin injections and oral medication(s), and about 10% of individuals may use diet and exercise alone.

Current therapies for diabetes mellitus include: insulin; insulin secretagogues, such as sulphonylureas, which increase insulin production from pancreatic 13-cells; glucose-lowering effectors, such as metformin which reduce glucose production from the liver; activators of the peroxisome proliferator-activated receptor-γ (PPAR-γ), such as the thiazolidinediones, which enhances insulin action; and α-glucosidase inhibitors which interfere with gut glucose production. There are, however, deficiencies associated with currently available treatments, including hypoglycemic episodes, weight gain, loss in responsiveness to therapy over time, gastrointestinal problems, and edema.

There are several areas at which research is being targeted in order to bring new, more effective, therapies to the marketplace. For example, on-going research includes exploring a reduction in excessive hepatic glucose production, enhancing the pathway by which insulin transmits its signal to the cells such that they take up glucose, enhancing glucose-stimulated insulin secretion from the pancreatic 13-cells, and targeting obesity and associated problems with fat metabolism and accumulation.

One particular target is GPR119. GPR119 is a member of the rhodopsin family of G-protein-coupled receptors. In addition to the “GPR119” identifier, several other identifiers exist, including but not limited to RUP 3, Snorf 25, 19 AJ, GPR 116 (believed to be erroneous), AXOR 20, and PS1. GPR119 is expressed in human gastrointestinal regions and in human islets. Activation of GPR119 has been demonstrated to stimulate intracellular cAMP and lead to glucose-dependent GLP-1 and insulin secretion. See, T. Soga et al., Biochemical and Biophysical Research Communications 326 (2005) 744-751, herein incorporated by reference with regard to a background understanding of GPR119.

In type 2 diabetes the action of GLP-1 on the β-cell is maintained, although GLP-1 secretion, itself, is reduced. More recently, therefore, much research has been focused on GLP-1. Studies show glucose-lowering effects in addition to GLP-1's ability to stimulate glucose-dependent insulin secretion including, but not limited to, an inhibition of the release of the hormone glucagon following meals, a reduction in the rate at which nutrients are absorbed into the bloodstream, and a reduction of food intake. Studies demonstrate that treatments to increase GLP-1, therefore, may be used for a variety of conditions and disorders including but not limited to metabolic disorders, gastrointestinal disorders, inflammatory diseases, psychosomatic, depressive, and neuropsychiatric disease including but not limited to diabetes mellitus (Type 1 and Type 2), metabolic syndrome, obesity, appetite control and satiety, weight loss, stress, inflammation, myocardial ischemia/reperfusion injury, Alzheimer's Disease, and other diseases of the central nervous system.

The use of exogenous GLP-1 in clinical treatment is severely limited, however, due to its rapid degradation by the protease DPP-IV. There are multiple GLP-1 mimetics in development for type 2 diabetes that are reported in the literature, all are modified peptides, which display longer half-lives than endogenous GLP-1. For example, the product sold under the tradename BYETTA® is the first FDA-approved agent of this new class of medications. These mimetics, however, require injection. An oral medication that is able to elevate GLP-1 secretion is desirable. Orally available inhibitors of DPP-IV, which result in elevation in intact GLP-1, are now available, such as sitagliptin, marketed under the brand name JANUVIA®. Nevertheless, a molecule which may stimulate GLP-1 secretion would provide a therapeutic benefit. A molecule which could stimulate both GLP-1 secretion and insulin secretion through effects on the L-cell and direct effects on the β-cell would hold much promise for type 2 diabetes therapy.

The present invention identifies agonists of GPR119 which increase glucose-disposal in part through elevation of GIP, GLP-1, and insulin. Moreover, studies demonstrate that GPR119 agonists such as the compounds of the present invention can stimulate incretins independently of glucose. GIP and GLP-1 are peptides, known as incretins, secreted from enteroendocrine K and L cells, respectively, in response to ingestion of nutrients, and have a wide variety of physiological effects that have been described in numerous publications over the past two decades. See, for example, Bojanowska, E. et al., Med. Sci. Monit., 2005, August 11(8): RA271-8; Perry, T. et al., Curr. Alzheimer Res., 2005, July 2(3): 377-85; and Meier, J. J. et al., Diabetes Metab. Res. Rev., 2005, March-April; 21(2); 91-117 (each herein incorporated by reference with regard to a background understanding of incretins). Moreover, although the mechanisms regulating GLP-1 secretion remain unclear, the initial rapid rise in GLP-1 following a meal may be a result of hormonal stimulation of neuronal afferents involving GIP. See, for example, J. N. Roberge and P. L. Brubaker, Endocrinology 133 (1993), pp. 233-240 (herein incorporated by reference with regard to such teaching). Furthermore, later increases in GLP-1 may involve direct activation of L-cells by nutrients in the distal small-intestine and the colon. GIP and GLP-1 are potent stimulators of the body's ability to produce insulin in response to elevated levels of blood sugar. In Type 2 diabetes, patients display a decreased responsiveness to GIP but not GLP-1, with respect to its ability to stimulate insulin secretion. The mechanism behind the decreased responsiveness to GIP remains unclear since type 2 diabetics retain sensitivity to a bolus administration of GIP but not to a continuous infusion (Meier et al. 2004 Diabetes 53 S220-S224). Moreover recent studies with a long-acting fatty-acid derivative of GIP showed beneficial effects on glucose homeostasis in ob/ob mice following 14 days of treatment (Irwin N. et al. (2006) J. Med. Chem. 49, 1047-1054.)

Agonists to GPR119 may be of therapeutic value for diabetes and associated conditions, particularly type II diabetes, obesity, glucose intolerance, insulin resistance, metabolic syndrome X, hyperlipidemia, hypercholesterolemia, and atherosclerosis.

Pyridazines are disclosed in U.S. Pat. No. 5,231,184, including compound numbers 145, 152, 153 and 163 having 4 rings.

### SUMMARY OF THE PRESENT INVENTION

There is provided a compound of the formula (I) or a pharmaceutically acceptable salt thereof:

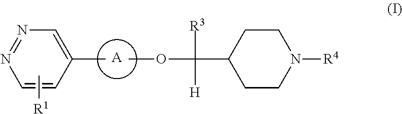

wherein
R¹ is selected from the group consisting of —C₁₋₃alkyl or halogen;
ring A is selected from the group consisting of:

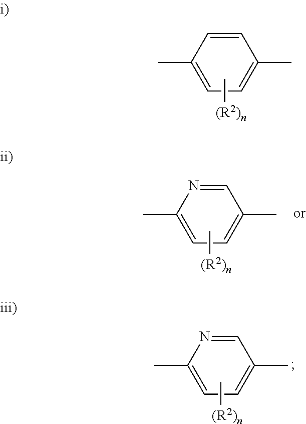

R² is a replacement for a hydrogen atom and is independently selected from the group consisting of halogen, —CF₃, —OH, —C₁₋₅alkyl, —C₃₋₇cycloalkyl, and —C₁₋₅alkoxyl;
n is 0, 1, or 2;
R³ is selected from a group consisting of —H, —C₁₋₅alkyl, or —C₃₋₇cycloalkyl;
R⁴ is —C(O)C(O)R⁵, —C(O)OR⁵, —C(O)R⁵, —S(O)₂C₁₋₅alkyl, —S(O)₂C₃₋₇cycloalkyl, —S(O)₂NR⁶R⁷, —Ar, —CH₂Ar, —C(O)NHC₁₋₅alkyl, —C(O)NHC₃₋₇cycloalkyl, —C(O)NHC₁₋₅alkyl-Ar, or —C(O)NR⁶R⁷;
R⁵ is independently selected from the group consisting of
   —C₁₋₅alkyl,
   —C₃₋₇cycloalkyl,
   phenyl,
   phenyl(C₁₋₄alkylene),
   a heterocyclic group of 3-7 ring members, and
   —C₁₋₅alkyl substituted by a heterocyclic group of 3-7 ring members,
which group members may be further optionally substituted by one or more of halogen, —C₁₋₅alkoxyl, a heteroaryl ring of 5-6 members,
—NR⁶R⁷, or —C(O)NR⁶R⁷;
R⁶ and R⁷ are independently selected from the group consisting of —H, —C₁₋₅alkyl, —C₃₋₇cycloalkyl, and a heterocyclic group of 3-7 members or R⁶ and R⁷ are alkyl and together combine to form a ring having 4 to 7 ring atoms and optionally containing a heterogroup selected from —O—, —NH—, and —N(C₁₋₅alkyl)- and wherein said ring having 4 to 7 ring atoms is optionally substituted by oxo; and
Ar is aryl or a 5- or 6-membered heteroaryl group, which may be substituted by one or more substituents independently selected from halogen, —CF₃, —C₁₋₅alkyl, —C₃₋₇cycloalkyl, —CN, —OR⁵, —NR⁶R⁷, and —NO₂.

Embodiments of the invention include a pharmaceutical composition comprising a compound of the present invention for use as an active therapeutic substance.

An aspect of the invention is a compound of the invention for use in the treatment (including prophylaxis) of diseases and conditions mediated through GPR119.

An aspect of the invention is a compound of the invention for use in the treatment (including prophylaxis) of metabolic disorders or conditions, such as diabetes and/or obesity.

An aspect of the invention is the use a compound of the invention in the manufacture of a medicament for use in the treatment (including prophylaxis) of metabolic disorders or conditions, such as diabetes and/or obesity.

An aspect of the invention is a method for the treatment (including prophylaxis) of metabolic disorders or conditions, such as diabetes or obesity, comprising the administration of a compound of the invention.

One embodiment of the invention is a method for increasing GLP-1 secretion in a glucose independent and dependent manner through the administration of a GPR119 agonist, such as a compound of the invention.

One embodiment of the invention is a method for reducing food intake through the administration of a GPR119 agonist, such as a compound of the invention.

The present invention covers all combinations of particular and preferred groups herein described.

### DETAILED DESCRIPTION OF THE PREFERRED EMBODIMENTS

A compound of the formula (I) or a pharmaceutically acceptable salt thereof:

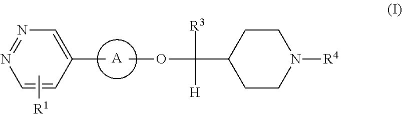

wherein
R¹ is selected from the group consisting of —C₁₋₃alkyl or halogen;
ring A is selected from the group consisting of:

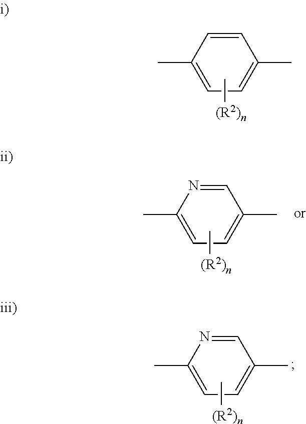

R² is a replacement for a hydrogen atom and is independently selected from the group consisting of halogen, —CF₃, —OH, —C₁₋₅alkyl, —C₃₋₇cycloalkyl, and —C₁₋₅alkoxyl;
n is 0, 1, or 2;
R³ is selected from a group consisting of —H, —C₁₋₅alkyl, or —C₃₋₇cycloalkyl;
R⁴ is —C(O)C(O)R⁵, —C(O)OR⁵, —C(O)R⁵, —S(O)₂C₁₋₅alkyl, —S(O)₂C₃₋₇cycloalkyl, —S(O)₂NR⁶R⁷, —Ar, —CH₂Ar, —C(O)NHC₁₋₅alkyl, —C(O)NHC₃₋₇cycloalkyl, —C(O)NHC₁₋₅alkyl-Ar, or —C(O)NR⁶R⁷;
R⁵ is independently selected from the group consisting of
   —C₁₋₅alkyl,
   —C₃₋₇cycloalkyl,
   phenyl,
   phenyl(C₁₋₄alkylene),
   a heterocyclic group of 3-7 ring members, and
   —C₁₋₅alkyl substituted by a heterocyclic group of 3-7 ring members,
which group members may be further optionally substituted by one or more of halogen, —C₁₋₅alkoxyl, a heteroaryl ring of 5-6 members,
—NR⁶R⁷, or —C(O)NR⁶R⁷;
R⁶ and R⁷ are independently selected from the group consisting of —H, —C₁₋₅alkyl, —C₃₋₇cycloalkyl, and a heterocyclic group of 3-7 members or R⁶ and R⁷ are alkyl and together combine to form a ring having 4 to 7 ring atoms and optionally containing a heterogroup selected from —O—, —NH—, and —N(C₁₋₅alkyl)- and wherein said ring having 4 to 7 ring atoms is optionally substituted by oxo; and
Ar is aryl or a 5- or 6-membered heteroaryl group, which may be substituted by one or more substituents independently selected from halogen, —CF₃, —C₁₋₅alkyl, —C₃₋₇cycloalkyl, —CN, —OR⁵, —NR⁶R⁷, and —NO₂.

In one embodiment of formula (I), R³ is —CH₃. In a preferred embodiment of formula (I), R³ is —CH₃ and the stereochemistry of the stereogenic carbon is (S).

In an embodiment of formula (I), R⁴ is —C(O)OR⁵ and R⁵ is selected from the group consisting of: —C₁₋₅alkyl and —C₃₋₇cycloalkyl.

In one embodiment of formula (I) ring A is:

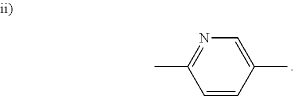

And in another embodiment of formula (I) ring A is:

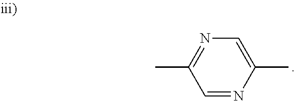

The present invention is described in terms known and appreciated by those skilled in the art. For ease of reference certain terms hereinafter are defined. The fact that certain terms are defined, however, should not be considered as indicative that defined terms are used in a manner inconsistent with the ordinary meaning or, alternatively, that any term that is undefined is indefinite or not used within the ordinary and accepted meaning. Rather, all terms used herein are believed to describe the invention such that one of ordinary skill can appreciate the scope of the present invention. The following definitions are meant to clarify, but not limit, the terms defined.

“Alkyl” refers to a monovalent straight or branched chain hydrocarbon moiety, e.g. of about 1 to 12 carbon atoms, including methyl, ethyl, n-propyl, isopropyl, isobutyl, n-butyl, tert-butyl, isopentyl and n-pentyl.

A specific number of atoms in a group, such as carbon atoms, will be represented by, for example, the phrase “Cₓ-C_{y} alkyl,” which refers to an alkyl group, containing the specified number of carbon atoms.

“Alkenyl” refers to a monovalent straight or branched chain aliphatic hydrocarbon moiety, e.g. of about 1 to 12 carbons, containing one or more carbon-to-carbon double bonds, such as vinyl and allyl.

“Alkylene” refers to a divalent straight or branched chain aliphatic hydrocarbon moiety, e.g. of about 1 to 10 carbon atoms, including methylene, ethylene, n-propylene, and n-butylene.

“Cycloalkyl” refers to a monovalent aliphatic cyclic hydrocarbon ring moiety, e.g. of about 1 to 12 carbons, including cyclopropyl, cyclobutyl, cyclopentyl, cyclohexyl, and cycloheptyl. The term “cycloalkyl” includes a fused ring system where a cycloalkyl ring, such as a cyclopentyl ring, is fused with an aromatic ring, herein an aryl ring, such as a benzene ring, to form groups such as indane.

“Heterocyclic” refers to a monovalent mono- or polycyclic ring system, e.g. of about 3 to 12 members, which may be aromatic, have no unsaturation, or may contain one or more degrees of unsaturation, containing 1 or more heteroatoms including N, O, and/or S, including N-oxides, sulfur oxides, and dioxides. Such rings may be fused to one or more of another heterocyclic ring(s) or cycloalkyl ring(s). Such fused ring systems include a saturated heterocyclic ring (such as a pyrrolidine ring) fused with an aromatic ring, such as a benzene ring to form groups such as indoline. Examples of heterocyclic groups include tetrahydrofuran, pyran,1,4-dioxane, 1,3-dioxane, piperidine, pyridine, pyrrolidine, morpholine, tetrahydrothiopyran, and tetrahydrothiophene.

“Aryl” refers to a monovalent benzene ring or to a fused benzene ring system, e.g. of about 6 to 14 carbons, such as anthracene, phenanthrene, or naphthalene ring systems, including phenyl, 2-naphthyl and 1-naphthyl.

“Heteroaryl” refers to a monovalent aromatic monocyclic ring, e.g. of 5 to 7 members, or to a fused bicyclic aromatic ring system comprising two aromatic rings that contain one or more N, S, and/or O atoms, including N-oxides, sulfur oxides, and dioxides, including furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, benzimidizolyl, imidazopyridinyl, pyrazolopyridinyl and pyrazolopyrimidinyl.

“Alkoxy” and “alkoxyl” refers to a monovalent group —O-alkyl.

“Halogen” refers to fluorine, chlorine, bromine, or iodine.

R¹, in particular, may be —CH₃ or halogen.

Attachment of Ring A in the compounds of formula (I) is as depicted in the formulae herein.

R², in particular, may be —F, —OCH₃ or —CH₃ with n, in particular, being 0, 1 or 2.

R³, in particular, may be H or —CH₃.

R⁴, in particular, may be —C(O)OCH(CH₃)₂, —C(═N—)(—O—N═)C—CH(CH₃)₂.

Compounds of formula (I) may crystallize in more than one form, a characteristic known as polymorphism, and such polymorphic forms (“polymorphs”) are within the scope of compounds of the invention. Polymorphism generally can occur as a response to changes in temperature, pressure, or both, and can also result from variations in the crystallization process. Polymorphs can be distinguished by various physical characteristics such as x-ray diffraction patterns, solubility, and melting point.

Certain of the compounds described herein may be capable of existing as stereoisomers such as by having a chiral carbon, sulfoxide sulfur or double bond whereby the compounds may exist as R or S enantiomers or E or Z isomers. The scope of the present invention includes all such individual isomers, racemates, purified enantiomers, and enantiomerically enriched mixtures of the compounds of formula (I).

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

Included within the scope of the invention compounds are solvates of compounds of the depicted formula. “Solvate” refers to a complex of variable stoichiometry formed by a solute (in this invention, a compound of Formula (I), or a salt or physiologically functional derivative thereof) and a solvent. Such solvents, for the purpose of the invention, should not interfere with the biological activity of the solute. Preferably the solvent used is a pharmaceutically acceptable solvent such as water, ethanol, and acetic acid.

“Physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol. 1: Principles and Practice, which is incorporated herein by reference to the extent that it teaches physiologically functional derivatives.

“Effective amount” means that amount of a drug or pharmaceutical agent that will elicit the biological or medical response of a tissue, system, animal, or human that is being sought, for instance, by a researcher or clinician.

“Therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of formula (I), as well as salts, solvates, and physiological functional derivatives thereof, may be administered as the raw chemical. Additionally, the active ingredient may be presented as a pharmaceutical composition.

Accordingly, the invention further provides pharmaceutical compositions that include effective amounts of a compound of the formula (I) or a salt, solvate, or physiological functional derivative thereof, and one or more pharmaceutically acceptable carriers, diluents, or excipients. The carrier(s), diluent(s) or excipient(s) must be acceptable, in the sense of being compatible with the other ingredients of the formulation and not deleterious to the recipient of the pharmaceutical composition.

In another aspect of the invention there is provided a process for the preparation of a pharmaceutical formulation including admixing a compound of the formula (I) or a salt, solvate, or physiological functional derivative thereof, with one or more pharmaceutically acceptable carriers, diluents or excipients.

A therapeutically effective amount of a compound of the present invention will depend upon a number of factors. The species, age, and weight of the recipient, the precise condition requiring treatment and its severity, the nature of the formulation, and the route of administration are all factors to be considered. The therapeutically effective amount ultimately should be at the discretion of the attendant physician or veterinarian. An effective amount of a compound of formula (I) for the treatment of humans or other mammals suffering from metabolic disorders such as diabetes and obesity, generally, should be in the range of about 0.1 to 100 mg/kg body weight of recipient (mammal) per day. More usually the effective amount should be in the range of 0.1 to 10 mg/kg body weight per day. Thus, for a 70 kg adult mammal the actual amount per day would usually be from 7 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of formula (I) per se. Similar dosages should be appropriate for treatment of the other conditions referred to herein and for prophylaxis.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s).

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For oral administration in the form of a tablet or capsule, the active drug component can be combined with an oral, non-toxic pharmaceutically acceptable inert carrier such as ethanol, glycerol or water. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Suitable binders, lubricants, disintegrating agents, and coloring agents can also be incorporated into the mixture. Examples of binders include starch, gelatin, natural sugars such as glucose or beta-lactose, corn sweeteners, natural and synthetic gums such as acacia, tragacanth, or sodium alginate, carboxymethylcellulose, polyethylene glycol and waxes. Lubricants useful in these dosage forms include sodium oleate, sodium stearate, magnesium stearate, sodium benzoate, sodium acetate and sodium chloride. Disintegrators include starch, methyl cellulose, agar, bentonite and xanthan gum.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage, or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. Compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; can also be added.

Where appropriate, dosage unit formulations for oral administration can be microencapsulated. The formulation can also be prepared to prolong or sustain the release as for example by coating or embedding particulate material in polymers or wax.

Compounds of formula (I) and salts, solvates, and physiological functional derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of formula (I) and salts, solvates, and physiologically functional derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as described in Pharmaceutical Research, 3(6), 318 (1986).

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

Compounds of the present invention and their salts, solvates, and physiologically functional derivatives thereof, may be employed alone or in combination with other therapeutic agents. The compound(s) of formula (I) and the other pharmaceutically active agent(s) may be administered together or separately and, when administered separately, administration may occur simultaneously or sequentially, in any order. The amounts of the compound(s) of formula (I) and the other pharmaceutically active agent(s) and the relative timings of administration will be selected in order to achieve the desired combined therapeutic effect. The administration in combination of a compound of formula (I) salts, solvates, or physiologically functional derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

Compounds of the present invention may be used in the treatment of a variety of disorders and conditions. As such, the compounds of the present invention may be used in combination with a variety of other therapeutic agents useful in the treatment or prophylaxis of those disorders or conditions. The compounds of the present invention may be used in combination with diet, exercise, insulin, an insulin sensitivity enhancer, a glucose absorption inhibitor, a biguanide, an insulin secretion enhancer, a SGLT2 inhibitor, an insulin or insulin analogue, a glucagon receptor antagonist, an insulin receptor kinase stimulant, a tripeptidyl peptidase II inhibitor, a dipeptidyl peptidase IV inhibitor, a protein tyrosine phosphatase-1B inhibitor, a glycogen phosphorylase inhibitor, an AXOR 109 agonist, a glucose-6-phosphatase inhibitor, a fructose-bisphosphatase inhibitor, a pyruvate dehydrogenase inhibitor, a hepatic gluconeogenesis inhibitor, D-chiroinsitol, a glycogen synthase kinase-3 inhibitor, glucagon-like peptide-1, a glucagon-like peptide-1 analogue, a glucagon-like peptide-1 agonist, amylin, an amylin analogue, an amylin agonist, an aldose reductase inhibitor, an advanced glycation endproducts formation inhibitor, a protein kinase C inhibitor, a γ-aminobutyric acid receptor antagonist, a sodium channel antagonist, a transcript factor NF-κB inhibitor, a lipid peroxidase inhibitor, an N-acetylated-α-linked-acid-dipeptidase inhibitor, insulin-like growth factor-I, platelet-derived growth factor, a platelet-derived growth factor analogue, epidermal growth factor, nerve growth factor, a carnitine derivative, uridine, 5-hydroxy-1-methylhidantoin, EGB-761, bimoclomol, sulodexide, Y-128, antidiarrhoics, cathartics, a hydroxymethylglutaryl coenzyme A reductase inhibitor, a fibric acid derivative, a β₃-adrenoceptor agonist, an acyl-coenzyme A cholesterol acyltransferase inhibitor, probcol, a thyroid hormone receptor agonist, a cholesterol absorption inhibitor, a lipase inhibitor, a microsomal triglyceride transfer protein inhibitor, a lipoxygenase inhibitor, a carnitine palmitoyl-transferase inhibitor, a squalene synthase inhibitor, a low-density lipoprotein receptor enhancer, a nicotinic acid derivative, a bile acid sequestrant, a sodium/bile acid cotransporter inhibitor, a cholesterol ester transfer protein inhibitor, an appetite suppressant, an angiotensin-converting enzyme inhibitor, a neutral endopeptidase inhibitor, an angiotensin II receptor antagonist, an endothelin-converting enzyme inhibitor, an endothelin receptor antagonist, a diuretic agent, a calcium antagonist, a vasodilating antihypertensive agent, a sympathetic blocking agent, a centrally acting antihypertensive agent, an α₂-adrenoceptor agonist, an antiplatelets agent, a uric acid synthesis inhibitor, a uricosuric agent, and a urinary alkalinizer.

Exemplary compounds are hereinafter described, however, a combination within the scope of the present invention should not be limited by this specific description. Rather, any combination within the purview of those skilled in the art is contemplated. In addition, this listing of exemplary compounds includes the free compounds, as well as salts, solvates, and physiologically functional derivatives.

As insulin sensitivity enhancers, peroxisome proliferator-activated receptor-γ agonists such as troglitazone, pioglitazone, rosiglitazone, darglitazone, GI-262570, isaglitazone, LG-100641, NC-2100, T-174, DRF-2189, CLX-0921, CS-011, GW-1929, ciglitazone, englitazone, and NIP-221, peroxisome proliferator-activated receptor-α agonists such as GW-9578 and BM-170744, peroxisome proliferator-activated receptor-α/γ agonists such as GW-409544, KRP-297, N,N-622, CLX-0940, LR-90, SB-219994, DRF-4158, and DRF-MDX8, retinoid X receptor agonists such as ALRT-268, AGN-4204, MX-6054, AGN-194204, LG-100754 and bexarotene, and other insulin sensitivity enhancers such as reglixane, ONO-5816, MBX-102, CRE-1625, FK-614, CLX-0901, CRE-1633, N,N-2344, BM-13125, BM-501050, HQL-975, CLX-0900, MBX-668, MBX-675, S-15261, GW-544, AZ-242, LY-510929, AR-H049020 and GW-501516 are illustrated. Insulin sensitivity enhancers may be used for diabetes, impaired glucose tolerance, diabetic complications, obesity, hyperinsulinemia, hyperlipidemia, hypercholesterolemia, hypertriglyceridemia, lipid metabolism disorder or atherosclerosis, and more preferably for diabetes, impaired glucose tolerance or hyperinsulinemia. Such compounds are believed to improve the disturbance of insulin signal transduction in peripheral tissues and enhancing glucose uptake into the tissues from the blood, leading to lowering of blood glucose level.

As glucose absorption inhibitors, for example, α-glucosidase inhibitors such as acarbose, voglibose, miglitol, CKD-711, emiglitate, MDL-25,637, camiglibose and MDL-73,945, and α-amylase inhibitors such as AZM-127 are illustrated. Glucose absorption inhibitors may be used for diabetes, impaired glucose tolerance, diabetic complications, obesity or hyperinsulinemia, and more preferably for impaired glucose tolerance. Such compounds are believed to inhibit the gastrointestinal enzymatic digestion of carbohydrates contained in foods, and inhibit and/or delay the absorption of glucose into the body.

As biguanides, phenformin, buformin, metformin, or the like are illustrated. Biguanides may be used for diabetes, impaired glucose tolerance, diabetic complications or hyperinsulinemia, and more preferably for diabetes, impaired glucose tolerance or hyperinsulinemia. Such compounds are believed to lower blood glucose level by inhibitory effects on hepatic gluconeogenesis, accelerating effects on anaerobic glycolysis in tissues or improving effects on insulin resistance in peripheral tissues.

As insulin secretion enhancers, tolbutamide, chlorpropamide, tolazamide, acetohexamide, glyclopyramide, glyburide (glibenclamide), gliclazide, 1-butyl-3-metanilylurea, carbutamide, glibornuride, glipizide, gliquidone, glisoxapide, glybuthiazol, glybuzole, glyhexamide, sodium glymidine, glypinamide, phenbutamide, tolcyclamide, glimepiride, nateglinide, mitiglinide calcium hydrate, repaglinide or the like are illustrated. In addition, the insulin secretion enhancers include glucokinase activators such as RO-28-1675. Insulin secretion enhancers may be used for diabetes, impaired glucose tolerance or diabetic complications, and more preferably for diabetes or impaired glucose tolerance. Such compounds are believed to lower blood glucose level by acting on pancreatic β-cells and enhancing the insulin secretion.

As SGLT2 inhibitors, compounds described in Japanese patent publications Nos. Hei 10-237089 and 2001-288178, and International Publication Nos. WO01/16147, WO01/27128, WO01/68660, WO01/74834, WO01/74835, WO02/28872, WO02/36602, WO02/44192, WO02/53573, and WO 03/99836 are illustrated. In addition, inhibitors identified as GW869682 and GSK189075 are illustrated as well. SGLT2 inhibitors may be used for diabetes, impaired glucose tolerance, diabetic complications, obesity or hyperinsulinemia, and more preferably for diabetes, impaired glucose tolerance, obesity or hyperinsulinemia. Such compounds are believed to lower blood glucose level by inhibiting the reabsorption of glucose at the kidney's proximal tubule.

As insulin or insulin analogues, human insulin, animal-derived insulin, human or animal-derived insulin analogues or the like are illustrated. These preparations may be used for diabetes, impaired glucose tolerance or diabetic complications, and more preferably for diabetes or impaired glucose tolerance.

AXOR109, also known as TGR5, BG37, M-BAR, or hGPCR19, is a bile acid G-protein coupled receptor primarily expressed in monocytes/macrophages, lung, spleen, and the intestinal tract. AXOR109 agonists may be used for diabetes mellitus, stress, obesity, appetite control and satiety, Alzheimers, inflammation, and diseases of the central nervous system. AXOR109 agonists are believed to moderate blood glucose level by stimulating the release of GLP-1 from enteroendocrine cells.

As glucagon receptor antagonists, BAY-27-9955, NNC-92-1687 or the like are illustrated; as insulin receptor kinase stimulants, TER-17411, L-783281, KRX-613 or the like are illustrated; as tripeptidyl peptidase II inhibitors, UCL-1397 or the like are illustrated; as dipeptidyl peptidase IV inhibitors, vildagliptin, sitigliptin, denagliptin, saxagliptin, TSL-225, P-32/98 or the like are illustrated; as protein tyrosine phosphatase 1B inhibitors, PTP-112, OC-86839, PNU-177496 or the like are illustrated; as glycogen phosphorylase inhibitors, N,N-4201, CP-368296 or the like are illustrated; as fructose-bisphosphatase inhibitors, R-132917 or the like are illustrated; as pyruvate dehydrogenase inhibitors, AZD-7545 or the like are illustrated; as hepatic gluconeogenesis inhibitors, FR-225659 or the like are illustrated; as glucagon-like peptide-1 analogues, exendin-4, CJC-1131 or the like are illustrated; as glucagon-like peptide 1 agonists; AZM-134, LY-315902 or the like are illustrated; and as amylin, amylin analogues or amylin agonists, pramlintide acetate or the like are illustrated. These drugs, glucose-6-phosphatase inhibitors, D-chiroinsitol, glycogen synthase kinase-3 inhibitors and glucagon-like peptide-1 may be used for diabetes, impaired glucose tolerance, diabetic complications or hyperinsulinemia, and more preferably for diabetes or impaired glucose tolerance.

As aldose reductase inhibitors, ascorbyl gamolenate, tolrestat, epalrestat, ADN-138, BAL-AR18, ZD-5522, ADN-311, GP-1447, IDD-598, fidarestat, sorbinil, ponalrestat, risarestat, zenarestat, minalrestat, methosorbinil, AL-1567, imirestat, M-16209, TAT, AD-5467, zopolrestat, AS-3201, NZ-314, SG-210, JTT-811, lindolrestat or the like are illustrated. Aldose reductase inhibitors may be used for diabetic complications. Such compounds are believed to inhibit aldose reductase and lowering excessive intracellular accumulation of sorbitol in accelated polyol pathway which are in continuous hyperglycemic condition in the tissues in diabetic complications.

As advanced glycation endproducts formation inhibitors, pyridoxamine, OPB-9195, ALT-946, ALT-711, pimagedine hydrochloride or the like are illustrated. Advanced glycation endproducts formation inhibitors may be used for diabetic complications. Such compounds are believed to inhibit formation of advanced glycation endproducts which are accelated in continuous hyperglycemic condition in diabetes and declining of cellular damage.

As protein kinase C inhibitors, LY-333531, midostaurin or the like are illustrated. Protein kinase C inhibitors may be used for diabetic complications. Such compounds are believed to inhibit protein kinase C activity, which is accelated in continuous hyperglycemic condition in diabetic patients.

As γ-aminobutyric acid receptor antagonists, topiramate or the like are illustrated; as sodium channel antagonists, mexiletine hydrochloride, oxcarbazepine or the like are illustrated; as transcrit factor NF-κB inhibitors, dexlipotam or the like are illustrated; as lipid peroxidase inhibitors, tirilazad mesylate or the like are illustrated; as N-acetylated-α-linked-acid-dipeptidase inhibitors, GPI-5693 or the like are illustrated; and as carnitine derivatives, carnitine, levacecamine hydrochloride, levocarnitine chloride, levocarnitine, ST-261 or the like are illustrated. These drugs, insulin-like growth factor-I, platelet-derived growth factor, platelet derived growth factor analogues, epidermal growth factor, nerve growth factor, uridine, 5-hydroxy-1-methylhidantoin, EGB-761, bimoclomol, sulodexide and Y-128 may be used for diabetic complications.

As antidiarrhoics or cathartics, polycarbophil calcium, albumin tannate, bismuth subnitrate or the like are illustrated. These drugs may be used for diarrhea, constipation or similar conditions that may accompany diabetes or other metabolic disorders.

As hydroxymethylglutaryl coenzyme A reductase inhibitors, sodium cerivastatin, sodium pravastatin, lovastatin, simvastatin, sodium fluvastatin, atorvastatin calcium hydrate, SC-45355, SQ-33600, CP-83101, BB-476, L-669262, S-2468, DMP-565, U-20685, BAY-x-2678, BAY-10-2987, calcium pitavastatin, calcium rosuvastatin, colestolone, dalvastatin, acitemate, mevastatin, crilvastatin, BMS-180431, BMY-21950, glenvastatin, carvastatin, BMY-22089, bervastatin or the like are illustrated. Hydroxymethylglutaryl coenzyme A reductase inhibitors may be used for hyperlipidemia, hypercholesterolemia, hypertriglyceridemia, lipid metabolism disorder or atherosclerosis, and more preferably for hyperlipidemia, hypercholesterolemia, or atherosclerosis. Such compounds are believed to lower blood cholesterol level by inhibiting hydroxymethylglutaryl coenzyme A reductase.

As fibric acid derivatives, bezafibrate, beclobrate, binifibrate, ciprofibrate, clinofibrate, clofibrate, aluminum clofibrate, clofibric acid, etofibrate, fenofibrate, gemfibrozil, nicofibrate, pirifibrate, ronifibrate, simfibrate, theofibrate, AHL-157 or the like are illustrated. Fibric acid derivatives may be used for hyperinsulinemia, hyperlipidemia, hypercholesterolemia, hypertriglyceridemia, lipid metabolism disorder or atherosclerosis, and more preferably for hyperlipidemia, hypertriglyceridemia, or atherosclerosis. Such compounds are believed to activate hepatic lipoprotein lipase and enhancing fatty acid oxidation, leading to a lowering of blood triglyceride levels.

As β₃-adrenoceptor agonists, BRL-28410, SR-58611A, ICI-198157, ZD-2079, BMS-194449, BRL-37344, CP-331679, CP-114271, L-750355, BMS-187413, SR-59062A, BMS-210285, LY-377604, SWR-0342SA, AZ-40140, SB-226552, D-7114, BRL-35135, FR-149175, BRL-26830A, CL-316243, AJ-9677, GW-427353 (solabegron), N-5984, GW-2696, YM178 or the like are illustrated. β₃-adrenoceptor agonists may be used for diabetes, obesity, hyperinsulinemia, hyperlipidemia, hypercholesterolemia, hypertriglyceridemia, lipid metabolism disorder, urinary incontinence, and IBS.

As acyl-coenzyme A cholesterol acyltransferase inhibitors, NTE-122, MCC-147, PD-132301-2, DUP-129, U-73482, U-76807, RP-70676, P-06139, CP-113818, RP-73163, FR-129169, FY-038, EAB-309, KY-455, LS-3115, FR-145237, T-2591, J-104127, R-755, FCE-28654, YIC-C8-434, avasimibe, CI-976, RP-64477, F-1394, eldacimibe, CS-505, CL-283546, YM-17E, lecimibide, 447C88, YM-750, E-5324, KW-3033, HL-004, eflucimibe or the like are illustrated. Acyl-coenzyme A cholesterol acyltransferase inhibitors may be used for hyperlipidemia, hypercholesterolemia, hypertriglyceridemia or lipid metabolism disorder, and more preferably for hyperlipidemia or hypercholesterolemia. Such compounds are believed to lower blood cholesterol levels by inhibiting acyl-coenzyme A cholesterol acyltransferase.

As thyroid hormone recptor agonists, sodium liothyronine, sodium levothyroxine, KB-2611 or the like are illustrated; as cholesterol absorption inhibitors, ezetimibe, SCH-48461 or the like are illustrated; as lipase inhibitors, orlistat, ATL-962, AZM-131, RED-103004 or the like are illustrated; as carnitine palmitoyltransferase inhibitors, etomoxir or the like are illustrated; as squalene synthase inhibitors, SDZ-268-198, BMS-188494, A-87049, RPR-101821, ZD-9720, RPR-107393, ER-27856 or the like are illustrated; as nicotinic acid derivatives, nicotinic acid, nicotinamide, nicomol, niceritrol, acipimox, nicorandil or the like are illustrated; as bile acid sequestrants, colestyramine, colestilan, colesevelam hydrochloride, GT-102-279 or the like are illustrated; as sodium/bile acid cotransporter inhibitors, 264W94, S-8921, SD-5613 or the like are illustrated; and as cholesterol ester transfer protein inhibitors, PNU-107368E, SC-795, JTT-705, CP-529414 or the like are illustrated. Probcol, microsomal trigylceride transfer protein inhibitors, lipoxygenase inhibitors, and low-density lipoprotein receptor enhancers may be used for hyperlipidemia, hypercholesterolemia, hypertrigly ceridemia, or lipid metabolism disorder.

As appetite suppressants, monoamine reuptake inhibitors, serotonin reuptake inhibitors, serotonin releasing stimulants, serotonin agonists (especially 5HT_{2c}-agonists), noradrenaline reuptake inhibitors, noradrenaline releasing stimulants, α₁-adrenoceptor agonists, β₂-adrenoceptor agonists, dopamine agonists, cannabinoid receptor antagonists, γ-aminobutyric acid receptor antagonists, H₃-histamine antagonists, L-histidine, leptin, leptin analogues, leptin receptor agonists, melanocortin receptor agonists (especially, MC3-R agonists, MC4-R agonists), α-melanocyte stimulating hormone, cocaine- and amphetamine-regulated transcript, mahogany protein, enterostatin agonists, calcitonin, calcitonin-gene-related peptide, bombesin, cholecystokinin agonists (especially CCK-A agonists), corticotropin-releasing hormone, corticotrophin-releasing hormone analogues, corticotropin-releasing hormone agonists, urocortin, somatostatin, somatostatin analogues, somatostatin receptor agonists, pituitary adenylate cyclase-activating peptide, brain-derived neurotrophic factor, ciliary neurotrophic factor, thyrotropin-releasing hormone, neurotensin, sauvagine, neuropeptide Y antagonists, opioid peptide antagonists, galanin antagonists, melanin-concentrating hormone antagonists, agouti-related protein inhibitors and orexin receptor antagonists are illustrated. As monoamine reuptake inhibitors, mazindol or the like are illustrated; as serotonin reuptake inhibitors, dexfenfluramine hydrochloride, fenfluramine, sibutramine hydrochloride, fluvoxamine maleate, sertraline hydrochloride or the like are illustrated; as serotonin agonists, inotriptan, (+)-norfenfluramine or the like are illustrated; as noradrenaline reuptake inhibitors, bupropion, GW-320659 or the like are illustrated; as noradrenaline releasing stimulants, rolipram, YM-992 or the like are illustrated; as β₂-adrenoceptor agonists, amphetamine, dextroamphetamine, phentermine, benzphetamine, methamphetamine, phendimetrazine, phenmetrazine, diethylpropion, phenylpropanolamine, clobenzorex or the like are illustrated; as dopamine agonists, ER-230, doprexin, bromocriptine mesylate or the like are illustrated; as cannabinoid receptor antagonists, rimonabant or the like are illustrated; as γ-aminobutyric acid receptor antagonists, topiramate or the like are illustrated; as H₃-histamine antagonists, GT-2394 or the like are illustrated; as leptin, leptin analogues or leptin receptor agonists, LY-355101 or the like are illustrated; as cholecystokinin agonists (especially CCK-A agonists), SR-146131, SSR-125180, BP-3.200, A-71623, FPL-15849, GI-248573, GW-7178, GI-181771, GW-7854, A-71378 or the like are illustrated; and as neuropeptide Y antagonists, SR-120819-A, PD-160170, NGD-95-1, BIBP-3226, 1229-U-91, CGP-71683, BIBO-3304, CP-671906-01, J-115814 or the like are illustrated.

As angiotensin-converting enzyme inhibitors, captopril, enalapri maleate, alacepril, delapril hydrochloride, ramipril, lisinopril, imidapril hydrochloride, benazepril hydrochloride, ceronapril monohydrate, cilazapril, sodium fosinopril, perindopril erbumine, calcium moveltipril, quinapril hydrochloride, spirapril hydrochloride, temocapril hydrochloride, trandolapril, calcium zofenopril, moexipril hydrochloride, rentiapril or the like are illustrated. Angiotensin-converting enzyme inhibitors may be used for diabetic complications or hypertension.

As neutral endopeptidase inhibitors, omapatrilat, MDL-100240, fasidotril, sampatrilat, GW-660511×, mixanpril, SA-7060, E-4030, SLV-306, ecadotril or the like are illustrated. Neutral endopeptidase inhibitors may be used for diabetic complications or hypertension.

As angiotensin II receptor antagonists, candesartan cilexetil, candesartan cilexetil/hydrochlorothiazide, potassium losartan, eprosartan mesylate, valsartan, telmisartan, irbesartan, EXP-3174, L-158809, EXP-3312, olmesartan, tasosartan, KT-3-671, GA-0113, RU-64276, EMD-90423, BR-9701 or the like are illustrated. Angiotensin II receptor antagonists may be used for diabetic complications or hypertension.

As endothelin-converting enzyme inhibitors, CGS-31447, CGS-35066, SM-19712 or the like are illustrated; as endothelin receptor antagonists, L-749805, TBC-3214, BMS-182874, BQ-610, TA-0201, SB-215355, PD-180988, sodium sitaxsentan, BMS-193884, darusentan, TBC-3711, bosentan, sodium tezosentan, J-104132, YM-598, S-0139, SB-234551, RPR-118031A, ATZ-1993, RO-61-1790, ABT-546, enlasentan, BMS-207940 or the like are illustrated. Such drugs may be used for diabetic complications or hypertension, and more preferably for hypertension.

As diuretic agents, chlorthalidone, metolazone, cyclopenthiazide, trichloromethiazide, hydrochlorothiazide, hydroflumethiazide, benzylhydrochlorothiazide, penflutizide, methyclothiazide, indapamide, tripamide, mefruside, azosemide, etacrynic acid, torasemide, piretanide, furosemide, bumetanide, meticrane, potassium canrenoate, spironolactone, triamterene, aminophylline, cicletanine hydrochloride, LLU-γ, PNU-80873A, isosorbide, D-mannitol, D-sorbitol, fructose, glycerin, acetazolamide, methazolamide, FR-179544, OPC-31260, lixivaptan, conivaptan hydrochloride or the like are illustrated. Diuretic drugs may be used for diabetic complications, hypertension, congestive heart failure or edema, and more preferably for hypertension, congestive heart failure or edema. Such compounds are believed to reduce blood pressure or improve edema by increasing urinary excretion.

As calcium antagonists, aranidipine, efonidipine hydrochloride, nicardipine hydrochloride, barnidipine hydrochloride, benidipine hydrochloride, manidipine hydrochloride, cilnidipine, nisoldipine, nitrendipine, nifedipine, nilvadipine, felodipine, amlodipine besilate, pranidipine, lercanidipine hydrochloride, isradipine, elgodipine, azelnidipine, lacidipine, vatanidipine hydrochloride, lemildipine, diltiazem hydrochloride, clentiazem maleate, verapamil hydrochloride, S-verapamil, fasudil hydrochloride, bepridil hydrochloride, gallopamil hydrochloride or the like are illustrated; as vasodilating antihypertensive agents, indapamide, todralazine hydrochloride, hydralazine hydrochloride, cadralazine, budralazine or the like are illustrated; as sympathetic blocking agents, amosulalol hydrochloride, terazosin hydrochloride, bunazosin hydrochloride, prazosin hydrochloride, doxazosin mesylate, propranolol hydrochloride, atenolol, metoprolol tartrate, carvedilol, nipradilol, celiprolol hydrochloride, nebivolol, betaxolol hydrochloride, pindolol, tertatolol hydrochloride, bevantolol hydrochloride, timolol maleate, carteolol hydrochloride, bisoprolol hemifumarate, bopindolol malonate, nipradilol, penbutolol sulfate, acebutolol hydrochloride, tilisolol hydrochloride, nadolol, urapidil, indoramin or the like are illustrated; as centrally acting antihypertensive agents, reserpine or the like are illustrated; and as α₂-adrenoceptor agonists, clonidine hydrochloride, methyldopa, CHF-1035, guanabenz acetate, guanfacine hydrochloride, moxonidine, lofexidine, talipexole hydrochloride or the like are illustrated. These drugs may be used for hypertension.

As antiplatelets agents, ticlopidine hydrochloride, dipyridamole, cilostazol, ethyl icosapentate, sarpogrelate hydrochloride, dilazep dihydrochloride, trapidil, beraprost sodium, aspirin or the like are illustrated. Antiplatelets agents may be used for atherosclerosis or congestive heart failure.

As uric acid synthesis inhibitors, allopurinol, oxypurinol or the like are illustrated; as uricosuric agents, benzbromarone, probenecid or the like are illustrated; and as urinary alkalinizers, sodium hydrogen carbonate, potassium citrate, sodium citrate or the like are illustrated. These drugs may be used for hyperuricemia or gout.

As noted, the compounds of the present invention may be used alone or may be combined with other medical therapies to treat and/or prevent a variety of disorders and conditions. More particularly, the diseases and conditions metabolic disorders, such as diabetes, including but not limited to diabetes types I and II, obesity, glucose intolerance, insulin resistance, metabolic syndrome X, hyperlipidemia, hypercholesterolemia, artheroscelrosis, neurodegenerative diseases, and other indications such as stroke.

Compounds of this invention may be made by a variety of methods. Illustrative general synthetic methods are set out below followed by a description of exemplary synthesis of specific compounds of the invention as illustrated in the examples.

In the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protective Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of formula (I).

Those skilled in the art will recognize if a stereocenter exists in compounds of formula (I). Accordingly, the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be affected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

The novel compounds of the present invention should not be limited by any specific synthetic process herein described.

### Experimental Section

The symbols and conventions used in the following descriptions of processes, schemes, and examples are consistent with those used in the contemporary scientific literature, for example, the Journal of the American Chemical Society or the Journal of Biological Chemistry.

Unless otherwise indicated, all temperatures are expressed in ° C. (degrees Centigrade). All reactions were conducted at room temperature unless otherwise noted. Unless otherwise indicated, definitions for moieties in formulae (II) through (VI) are as defined above for formula (I). Abbreviations and definitions include HPLC (high pressure liquid chromatography), LC-MS (liquid chromatography-mass spectrometry), NMR (nuclear magnetic resonance), TFA (trifluoroacetic acid); DIAD (diisopropylazodicarboxylate); IPA (isopropyl alcohol); DMSO (dimethylsulfoxide); THF (tetrahydrofuran); DMF (N,N-dimethylformamide); DME (1,2-dimethoxyethane); Ms (mesyl); Ts (tosyl); MTBE (methyl tert-butyl ether); DCM (dichloromethane); DBU (1,8-diazabicyclo[5.4.0]undec-7-ene); and aq (aqueous). All Rs (i.e., R¹, R², R³, R⁴, and so forth) are as previously defined in the specification unless defined otherwise in the schemes.

¹H-NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or bs (broad singlet).

Mass spectra were obtained on Acquity SQD or Micromass ZQ mass spectrometers from Waters Corporation, Milford, Mass., using either Atmospheric Chemical Ionization (APCI) or Electrospray Ionization (ESI).

### Synthetic Schemes

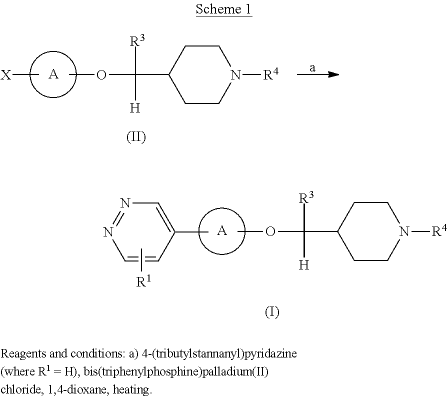

Biaryl-based compounds can be prepared by following the general synthetic scheme (Scheme 1). Aryl or heteroaryl halides (X=halogen, typically I, Br or Cl), of the general formula (II), can be coupled to tin reagents (such as 4-(tributylstannanyl)pyridazine) in the presence of a palladium catalyst such as bis(triphenylphosphine)palladium(II) chloride or by using procedures known to those skilled in the art. For a general reference for aryl-aryl bond formation, see: Hassan, J. et. al., Aryl-Aryl Bond Formation One Century after the Discovery of the Ullmann Reaction. Chemical Reviews, 2002, 102(5), 1359-1469. Synthesis of compounds of the general formula (II) has been described in WO/2008/070692 (PCT/US07/86434).

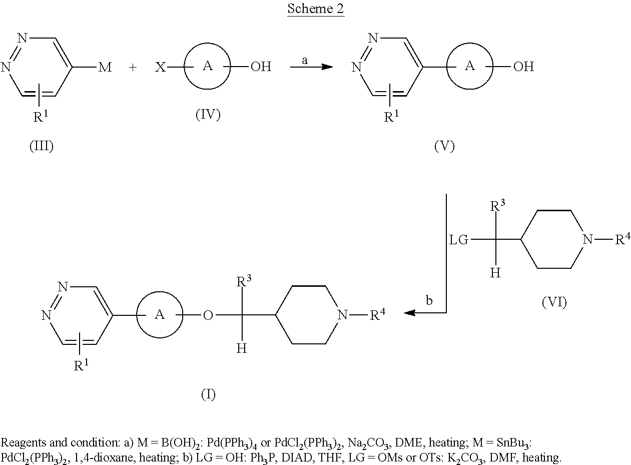

Alternatively, biaryl-based compounds can be prepared by following the general synthetic scheme (Scheme 2). The biaryl portion is coupled first using a Stille or Suzuki reaction under conditions a) between a substituted aryl tin reagent or aryl boronic acid (III), respectively, and an appropriately substituted aryl or heteroaryl halide (bromide or chloride) (IV) to provide intermediate (V). For Suzuki reaction conditions, see N. Miyaura and A. Suzuki, Chem. Rev., 1995, 95, 2457-2483; A. Suzuki, J. Organometallic Chem. 1999, 576, 147-168; and A. Suzuki, in Metal-catalyzed Cross-coupling Reactions, F. Diederich and P. J. Stang., Eds.; Wiley-VCH: New York, 1998, 49-97.

The compounds of formula (I) can be prepared from aryl alcohol or heteroaryl alcohol (V) and intermediate (VI) where LG- is HO— using Mitsunobu reaction conditions described in b), or from (V) and mesylate (VI) wherein LG- is mesyl in the presence of a suitable base such as K₂CO₃. For Mitsunobu reactions, see Mitsunobu, Synthesis, 1981, 1, and for a Mitsunobu reaction review see D. L. Hughes Organic Reactions 42, 335. For the formation of mesylate (VI) from its corresponding alcohol, see R. K. Crossland and K. L. Servis, J. Org. Chem., 1970, 35, 3195-3196. For reaction conditions for displacement of mesylate, see P. J. Gilligan, et al., J. Med. Chem., 1992, 35, 4344-4361. Compounds of the formula (VI) have been described in detail in WO/2008/070692 (PCT/US07/86434).

Compounds of formula (I), (II) or (VI) can also be prepared in enantioenriched fashion through chiral separation of racemic or enantioenriched material using, but not limited to, preparative chiral SFC technology. For a review, see: Christopher Welch, et al., LCGC North America January 2005, 23(1), 16-29.

In addition to the above general synthetic approaches and standard modifications thereto as known in the art, compounds of formula (I) can be obtained by reacting other compounds of formula (I) such as by conversion among the various R¹, R², R³, and R⁴ groups. For example, see Larock, R. C. In Comprehensive Organic Transformations: A Guide to Functional Group Preparations, VCH Publishers: New York, 1990. In the Schemes, R¹, R², R³, etc. are as defined in the specification unless otherwise noted.

Also within the scope of the invention are novel intermediates described above and in the Examples.

### EXAMPLES

The following specific examples are included as illustrations and are not to be construed as limiting the scope of the present invention.

### Example 1

### 1-Methylethyl 4-((1S)-1-{[5-(4-pyridazinyl)-2-pyrazinyl]oxy}ethyl)-1-piperidinecarboxylate

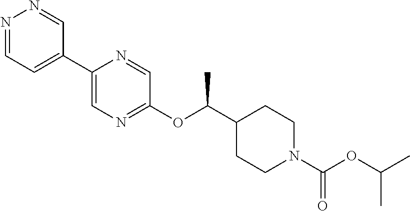

Step 1: Triethylamine (315 mL, 2.26 mol) was added dropwise to formic acid (150 mL, 3.91 mol) with overhead stirring while maintaining the internal temperature below 60° C. with ice-bath cooling. Neat 4-acetylpyridine (100 mL, 0.904 mol) was then added rapidly while maintaining the temperature below 50° C. Following this addition, the reaction was allowed to cool to 28° C. and the chiral ruthenium catalyst [N-[(1R,2R)-2-(amino-N)-1,2-diphenylethyl]-2,4,6-trimethylbenzenesulfonamidato-N]chloro[(1,2,3,4,5,6-n)-1-methyl-4-(1-methylethyl)benzene]ruthenium (CAS# 177552-91-9; for catalyst preparation, see: Uematsu, N.; Fujii, A.; Hashiguchi, S.; lkariya, T.; Noyori, R.; J. Am. Chem. Soc. 1996, 118, 4916-4917) (3 g, 4.46 mmol) was added. The mixture was stirred under house vacuum for 4 h and then overnight under an atmosphere of nitrogen. The reaction mixture was added dropwise to a stirred solution of 10% aq. Na₂CO₃ (4 L) and then extracted with EtOAc (3×1 L). The combined EtOAc layers were washed once with brine (1 L), treated with MgSO₄ and Darco G-60 decolorizing charcoal and filtered through a 100 g plug of silica gel washing with 10% MeOH/EtOAc (1 L). The filtrate was concentrated to provide a dark oil that crystallized upon standing. The solid was dissolved in warm t-butyl methyl ether (250 mL) and the warm solution was filtered to remove a small amount of insoluble material. The filtrate was allowed to stir with cooling to room temperature and then to −15° C. The solids were collected by filtration, washed with cold t-butyl methyl ether and heptane, and then dried under high vacuum to afford (1R)-1-(4-pyridinyl)ethanol as a dark beige solid (62 g, 52.9%). This solid material was 96% ee based on chiral HPLC (HPLC conditions: AS-H column, 5% MeOH/CO₂, 40° C., 140 bar, 2 mL/min). The filtrate was combined with the insoluble solid from the crystallization and concentrated in vacuo to afford additional (1R)-1-(4-pyridinyl)ethanol as a dark oil (37.5 g, 32%). This oily material was 78% ee based on chiral HPLC (see HPLC conditions above). ¹H NMR (400 MHz, DMSO-d₆): δ 8.47-8.43 (m, 2H), 7.32-7.28 (m, 2H), 5.37 (d, 1H, J=4.4 Hz), 4.72-4.64 (m, 1H), 1.44 (d, 3H, J=6.6 Hz).

Step 2: A solution of (1R)-1-(4-pyridinyl)ethanol (61 g, 0.495 mol, 96% ee) in MeOH (1.5 L) was charged with PtO₂ (15 g) under nitrogen atmosphere followed by acetic acid (40 mL). The mixture was evacuated and purged with hydrogen several times and then stirred under an atmosphere of hydrogen for 2 d at room temperature. The mixture was filtered to remove catalyst and the filtrate was concentrated in vacuo, charged with EtOAc (500 mL), and stirred at room temperature overnight. The solid was collected by filtration. The filter cake was washed with EtOAc (100 mL) twice, then dried under vacuum to afford (1R)-1-(4-piperidinyl)ethanol acetate (74.5 g, 79%) as a solid. ¹H NMR (400 MHz, DMSO-d₆): δ 3.35-3.23 (m, 1H), 3.03 (dd, 2H, J=12.5, 3.2 Hz), 2.58-2.42 (m, 3H), 1.77-1.65 (m, 4H), 1.51 (d, 1H, J=12.0 Hz), 1.34-1.07 (m, 3H), 0.97 (d, 3H, J=6.4 Hz).

Step 3: A suspension of (1R)-1-(4-piperidinyl)ethanol acetate (22.85 g, 0.121 mol) in dichloromethane (0.9 L) was treated with bis(1,1-dimethylethyl)dicarbonate (26.35 g, 0.121 mol). The mixture was treated dropwise with triethylamine (25.6 g, 0.254 mol), then warmed to a gentle reflux for 15 min. The mixture was cooled to room temperature, and then stirred for 4 h. The mixture was extracted with a 5% aq. NaHCO₃ solution (400 mL), then 10% aq. citric acid solution (400 mL), and finally with water (500 mL). The organic phase was dried over MgSO₄, filtered and concentrated to afford 1,1-dimethylethyl 4-[(1R)-1-hydroxyethyl]-1-piperidinecarboxylate (27.2 g, 98%) as a residue. ¹H NMR (400 MHz, CDCl₃): δ 4.13 (d, 2H, J=12.9 Hz), 3.71 (s, 1H), 3.56 (m, 1H), 2.63 (t, 2H, J=12.9 Hz), 1.79 (d, 1H, J=12.9 Hz), 1.62-1.53 (m, 1H), 1.48-1.33 (m, 12H), 1.16 (d, 3H, J=6.3 Hz).

Step 4: Sodium nitrite (1.082 g, 15.68 mmol) was added portionwise to concentrated H₂SO₄ (8 mL) at 0° C. The mixture was heated to 45° C. until all of the sodium nitrite dissolved, and was then cooled to 0° C. in an ice bath. A solution of 5-bromo-2-pyrazinamine (2 g, 11.5 mmol) in concentrated H₂SO₄ (12 mL) was added dropwise to the mixture. The ice bath was removed and the mixture was allowed to warm to 45° C. The mixture was stirred for 45 min at 45° C., and then slowly poured onto ice (200 g). An exotherm was observed and extra ice was added. The pH was adjusted to 3 with solid NaOH, and the mixture was extracted with EtOAc (80 mL, 4 times). The organics were combined, dried with MgSO₄, filtered and concentrated to afford 5-bromo-2-pyrazinol (and tautomers thereof) (1.53 g, 76%) as an off-white solid. ¹H NMR (400 MHz, CDCl₃): δ 8.06 (d, 1H, J=1.2 Hz), 7.60 (d, 1H, J=1.2 Hz). This sequence was repeated with 12.5 g of sodium nitrite, 200 mL concentrated H₂SO₄ and 23.2 g of 5-bromo-2-pyrazinamine to afford 17.7 g (76%) of 5-bromo-2-pyrazinol (and tautomers thereof). The batches were combined and used in the next step.

Step 5: 1,1-Dimethylethyl 4-[(1R)-1-hydroxyethyl]-1-piperidinecarboxylate (24.5 g, 0.107 mol), 5-bromo-2-pyrazinol (and tautomers thereof) (18.7 g, 0.107 mol), and triphenylphosphine (36.4 g, 0.139 mol) were dissolved THF (200 mL). A solution of DIAD (28.1 g, 0.139 mol) in THF (100 mL) was added to the mixture over 15 min during which time an exotherm was observed. The mixture stirred at room temperature for 45 min, and was concentrated by rotovap (keeping temperature <25° C.). The resulting residue was charged with hexanes-EtOAc (3:1), filtered, and the filtrate concentrated. The crude product was purified by chromatography on a silica gel column (750 g) eluted with 1:4 EtOAc/hexanes. The resultant material was subjected to analytical chiral HPLC [column: AD-H, mobile phase: 90% CO₂:10% IPA (2 mL/min), pressure 103 bar, 30° C.] and then separated using similar conditions [column: 3×25 cm AD-H, mobile phase: 85% CO₂:15% IPA (90 g/min), pressure 103 bar, 30° C.] to give 1,1-dimethylethyl 4-{(1S)-1-[(5-bromo-2-pyrazinyl)oxy]ethyl}-1-piperidinecarboxylate (first eluting peak, 19 g, 46%, 99% ee) as a brown oil. ¹H NMR (400 MHz, CDCl₃): δ 8.12 (d, 1H, J=1.2 Hz), 7.93 (s, 1H), 5.04-4.92 (m, 1H), 4.14 (d, 2H, J=1.7 Hz), 2.65 (t, 2H, J=12.3 Hz), 1.81-1.60 (m, 3H), 1.43 (s, 10H), 1.19 (d, 3H, J=6.1 Hz).

Step 6: A mixture of 1,1-dimethylethyl 4-{(1S)-1-[(5-bromo-2-pyrazinyl)oxy]ethyl}-1-piperidinecarboxylate (270 mg, 0.7 mmol), 4-(tributylstannanyl)pyridazine (672 mg, 1.75 mmol), and a catalytic amount of Pd(PPh₃)₂Cl₂ (10 mg) in 1,4-dioxane (10 mL) was heated at 110° C. under nitrogen overnight. The mixture was allowed to cool to room temperature, diluted with EtOAc, and washed with water, then brine. The organic phase was dried over Na₂SO₄, filtered and concentrated to afford a dark brown oil. The crude product was purified by flash chromatography (silica gel column, 0→100% EtOAc/hexane) to give a light brown oil. This material was further purified by chromatography (silica gel column, 20% acetone/CH₂Cl₂) to give 1,1-dimethylethyl 4-((1S)-1-{[5-(4-pyridazinyl)-2-pyrazinyl]oxy}ethyl)-1-piperidinecarboxylate (119 mg, 44%) as an off-white solid. ¹H NMR (400 MHz, CDCl₃): δ 9.75 (s, 1H), 9.26 (d, 1H, J=5.1 Hz), 8.64 (s, 1H), 8.28 (s, 1H), 8.07-7.94 (m, 1H), 5.14 (m, 1H), 4.33-4.00 (m, 2H), 2.79-2.56 (m, 2H), 1.90-1.62 (m, 3H), 1.44 (s, 9H), 1.39-1.18 (m, 5H); LRMS (ESI), m/z 386 (M+H).

Step 7: A solution of 1,1-dimethylethyl 4-((1S)-1-{[5-(4-pyridazinyl)-2-pyrazinyl]oxy}ethyl)-1-piperidinecarboxylate (0.106 g, 0.275 mmol) in CH₂Cl₂ (8 mL) was treated with TFA (0.214 mL, 2.75 mmol) and then stirred at room temperature overnight. The mixture was cooled to 0° C. in an ice bath, then charged with diisopropylethylamine (0.718 mL, 4.12 mmol), followed by a 1M solution of isopropyl chloroformate (0.33 mL, 0.33 mmol) in toluene. The mixture was allowed to warm to room temperature, and then stirred for 1 h. The mixture was diluted with CH₂Cl₂ (60 mL), washed with 1M NaH₂PO₄ (2×25 mL) and brine. The organic phase was dried over Na₂SO₄, filtered and concentrated to afford a yellow-brown oil. The crude product was purified by flash chromatography (silica gel column, 20% acetone/CH₂Cl₂) to give 75 mg (73%) of the title compound as an off-white solid after cooling with hexanes. ¹H NMR (400 MHz, CDCl₃): δ 9.76 (s, 1H), 9.27 (d, 1H, J=4.9 Hz), 8.64 (s, 1H), 8.28 (s, 1H), 8.05 (d, 1H, J=3.4 Hz), 5.14 (m, 1H), 4.89 (t, 1H, J=12.4 Hz), 4.20 (bs, 2H), 2.70 (t, 2H, J=12.6 Hz), 1.90-1.60 (m, 3H), 1.32 (m, 5H), 1.21 (d, 6H, J=6.2 Hz); LRMS (ESI), m/z 372 (M+H).

### Example 2

### 4-{5-[((1S)-1-{1-[3-(1-Methylethyl)-1,2,4-oxadiazol-5-yl]-4-piperidinyl}ethyl)oxy]-2-pyrazinyl}pyridazine

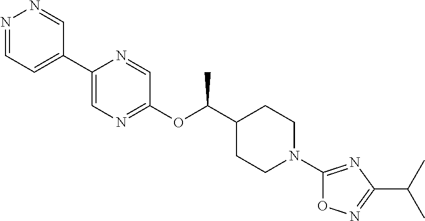

Step 1: A mixture of 1,1-dimethylethyl 4-((1S)-1-{[5-(4-pyridazinyl)-2-pyrazinyl]oxy}ethyl)-1-piperidinecarboxylate (prepared as in Example 1, Steps 1-6, 88 mg, 0.228 mmol) and TFA (0.176 mL, 2.283 mmol) in DCM (10 mL) was stirred at room temperature overnight. The mixture was concentrated to dryness, dissolved in 1:1 CH₂Cl₂:MeOH, and free-based with MP-Carbonate to give 4-(5-{[(1S)-1-(4-piperidinypethyl]oxy}-2-pyrazinyl)pyridazine (90 mg, 100%) as a yellow solid. ¹H NMR (400 MHz, CDCl₃): δ 9.83 (s, 1H), 9.34 (d, 1H, J=5.4 Hz), 8.68 (s, 1H), 8.32 (s, 1H), 8.18 (dd, 1H), 5.26-5.16 (m, 1H), 3.54 (d, 2H, J=12.2 Hz), 2.89 (d, 2H, J=9.9 Hz, 2H), 2.10-1.76 (m, 5H), 1.36 (d, 3H, J=6.2 Hz); LRMS (ESI), m/z 286 (M+H).

Step 2: DBU (0.143 mL, 0.949 mmol) was added to a mixture of 4-(5-{[(1S)-1-(4-piperidinypethyl]oxy}-2-pyrazinyl)pyridazine (90 mg, 0.315 mmol), 3-(1-methylethyl)-5-(trichloromethyl)-1,2,4-oxadiazole (see reference: WO 08070692, 362 mg, 1.58 mmol), and MeOH (1 mL) at room temperature. The mixture stirred at room temperature overnight. The mixture was concentrated to dryness. The resulting residue was purified by reverse-phase preparative HPLC using CH₃CN:H₂O gradient (10:90 to 100:0) with 0.05% TFA as a modifier. The resultant material was converted to its free base with MP-Carbonate to give the title compound (50 mg, 37%) as a white solid. ¹H NMR (400 MHz, CDCl₃): δ 9.75 (s, 1H), 9.27 (d, 1H, J=5.2 Hz), 8.63 (d, 1H, J=1.3 Hz), 8.29 (d, 1H, J=1.3 Hz), 8.01 (d, 1H, J=3.7 Hz), 5.18 (m, 1H), 4.25-4.15 (m, 2H), 3.09-2.98 (m, 2H), 2.94-2.79 (m, 1H), 1.96-1.76 (m, 3H), 1.56-1.38 (m, 2H), 1.34 (d, 3H, J=6.2 Hz), 1.26 (d, 6H, J=7.1 Hz); LRMS (ESI), m/z 396 (M+H).

### Example 3

### 2-Methylpropyl 4-((1S)-1-{[5-(4-pyridazinyl)-2-pyrazinyl]oxy}ethyl)-1-piperidinecarboxylate

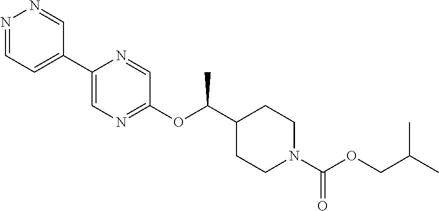

Step 1: A mixture of 1,1-dimethylethyl 4-((1S)-1-{[5-(4-pyridazinyl)-2-pyrazinyl]oxy}ethyl)-1-piperidinecarboxylate (prepared as in Example 1, Steps 1-6, 253 mg, 0.656 mmol), TFA (0.506 mL, 6.56 mmol), and DCM (10 mL) stirred at room temperature for 4 h. The mixture was concentrated to dryness to give 4-(5-{[(1S)-1-(4-piperidinypethyl]oxy}-2-pyrazinyl)pyridazine trifluoroacetate (400 mg, quant. yield) as a yellow oil. ¹H NMR (400 MHz, CDCl₃): δ 9.96 (s, 1H), 9.50 (d, 1H, J=5.6 Hz), 8.80 (s, 1H), 8.50 (d, 1H, J=3.9 Hz), 8.35 (s, 1H), 5.32-5.21 (m, 1H), 3.59 (d, 2H, J=12.2 Hz), 3.06-2.91 (m, 2H), 2.15-1.94 (m, 3H), 1.86-1.71 (m, 2H), 1.67-1.56 (m, 1H), 1.39 (d, 3H, J=6.4 Hz, 3H); LRMS (ESI), m/z 286 (M+H).

Step 2: 2-Methylpropyl chloridocarbonate (0.032 mL, 0.250 mmol) was added dropwise to a solution of 4-(5-{[(1S)-1-(4-piperidinypethyl]oxy}-2-pyrazinyl)pyridazine trifluoroacetate (100 mg, 0.250 mmol), Hunig's base (0.219 mL, 1.252 mmol), and dichloromethane (5 mL) at room temperature. The mixture stirred at room temperature for 1 h, and was then concentrated to dryness. The resulting residue was purified (twice) by reverse-phase preparative HPLC using CH₃CN:H₂O gradient (10:90 to 100:0) with 0.2% NH4OH as a modifier to give the title compound (6.8 mg, 7%) as a white solid.

¹H NMR (400 MHz, CDCl₃): δ 9.73 (d, 1H, J=1.1 Hz), 9.25 (d, 1H, J=4.9 Hz), 8.62 (d, 1H, J=1.5 Hz), 8.27 (d, 1H, J=1.3 Hz), 7.99-7.94 (m, 1H), 5.13 (m, 1H), 4.21 (d, 2H, J=0.9 Hz), 3.83 (d, 2H, J=6.7 Hz), 2.73 (bs, 2H), 1.96-1.86 (m, 1H), 1.86-1.66 (m, 3H), 1.36-1.24 (m, 5H), 0.90 (d, 6H J=6.9 Hz); LRMS (ESI), m/z 386 (M+H).

### Example 4

### Phenyl 4-((1S)-1-{[5-(4-pyridazinyl)-2-pyrazinyl]oxy}ethyl)-1-piperidinecarboxylate

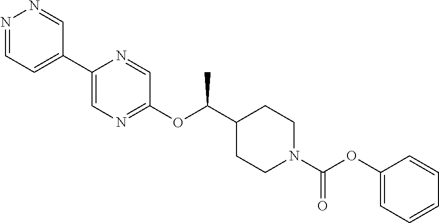

Phenyl chloridocarbonate (0.032 mL, 0.250 mmol) was added dropwise to a solution of 4-(5-{[(1S)-1-(4-piperidinypethyl]oxy}-2-pyrazinyl)pyridazine trifluoroacetate (prepared as in Example 3, Step 1, 100 mg, 0.250 mmol), Hunig's base (0.219 mL, 1.252 mmol), and DCM (5 mL) at room temperature. The mixture stirred at room temperature for 1 h, and was then concentrated to dryness. The resulting residue was purified by reverse-phase preparative HPLC using CH₃CN:H₂O gradient (10:90 to 100:0) with 0.2% NH4OH as a modifier to give the title compound (18 mg, 18%) as a white solid. ¹H NMR (400 MHz, CDCl₃): δ 9.75 (d, 1H, J=1.1 Hz), 9.26 (dd, 1H, J=5.5, 1.0 Hz), 8.64 (d, 1H, J=1.3 Hz), 8.30 (d, 1H, J=1.3 Hz), 8.04-7.96 (m, 1H), 7.33 (t, 2H, J=8.0 Hz), 7.16 (t, 1H, J=7.4 Hz), 7.10-7.05 (m, 2H), 5.19 (m, 1H), 4.35 (bs, 2H), 3.04-2.75 (m, 2H), 1.95-1.73 (m, 3H), 1.53-1.38 (m, 2H), 1.36 (d, 3H, J=6.2 Hz); LRMS (ESI), m/z 406 (M+H).

### Example 5

### 4-[5-({(1S)-1-[1-(5-Ethyl-2-pyrimidinyl)-4-piperidinyl]ethyl}oxy)-2-pyrazinyl]pyridazine

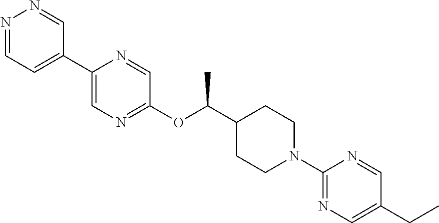

A mixture of 4-(5-{[(1S)-1-(4-piperidinypethyl]oxy}-2-pyrazinyl)pyridazine trifluoroacetate (prepared as in Example 3, Step 1, 100 mg, 0.250 mmol), 2-chloro-5-ethylpyrimidine (0.036 mL, 0.30 mmol) and potassium carbonate (69 mg, 0.50 mmol) in DMSO (2 mL) was stirred at 100° C. overnight. The mixture was filtered, and the resulting filtrate was purified by reverse-phase preparative HPLC using CH₃CN:H₂O gradient (10:90 to 100:0) with 0.05% TFA as a modifier. The resultant material was converted to its free base with MP-Carbonate to give the title compound (34 mg, 35%) as a white solid. ¹H NMR (400 MHz, CDCl₃): δ 9.73 (dd, 1H, J=2.3, 1.2 Hz), 9.24 (dd, 1H J=5.6, 1.1 Hz), 8.61 (d, 1H, J=1.3 Hz), 8.27 (d, 1H, J=1.3 Hz), 8.15 (s, 2H), 7.96 (dd, 1H, J=5.4, 2.4 Hz), 5.16 (m, 1H), 4.78 (dd, 2H, J=12.9, 1.5 Hz), 2.89-2.77 (m, 2H), 2.43 (q, 2H, J=7.6 Hz), 1.94-1.75 (m, 3H), 1.46-1.27 (m, 5H), 1.16 (t, 3H, J=7.6 Hz); LRMS (ESI), m/z 392 (M+H).

### Example 6

### 4-[5-({(1S)-1-[1-(5-Methyl-2-pyrimidinyl)-4-piperidinyl]ethyl}oxy)-2-pyrazinyl]pyridazine

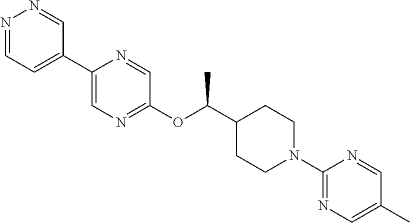

A mixture of 4-(5-{[(1S)-1-(4-piperidinypethyl]oxy}-2-pyrazinyl)pyridazine trifluoroacetate (prepared as in Example 3, Step 1, 63.5 mg, 0.159 mmol), 2-chloro-5-methylpyrimidine (25 mg, 0.19 mmol) and potassium carbonate (44 mg, 0.32 mmol) in DMSO (2 mL) was stirred at 100° C. overnight. The mixture was filtered, and the resulting filtrate was purified by reverse-phase preparative HPLC using CH₃CN:H₂O gradient (10:90 to 100:0) with 0.05% TFA as a modifier. The resultant material was repurified by reverse-phase preparative HPLC using CH₃CN:H₂O gradient (5:95 to 95:5) with 0.2% NH₄OH as a modifier to give the title compound (25 mg, 42%) as a white solid. ¹H NMR (400 MHz, CDCl₃): δ 9.73 (dd, 1H, J=2.4, 1.1 Hz), 9.24 (dd, 1H, J=5.5, 1.2 Hz), 8.61 (d, 1H, J=1.5 Hz), 8.27 (d, 1H, J=1.3 Hz), 8.13 (s, 2H), 8.00-7.92 (m, 1H), 5.21-5.10 (m, 1H), 4.77 (d, 2H, J=11.6 Hz), 2.83 (t, 2H, J=12.5 Hz), 2.09 (s, 3H), 1.95-1.86 (m, 2H), 1.83-1.74 (m, 1H), 1.46-1.28 (m, 5H); LRMS (ESI), m/z 378 (M+H).

### Example 7

### 4-[5-({(1S)-1-[1-(5-Chloro-2-pyrazinyl)-4-piperidinyl]ethyl}oxy)-2-pyrazinyl]pyridazine

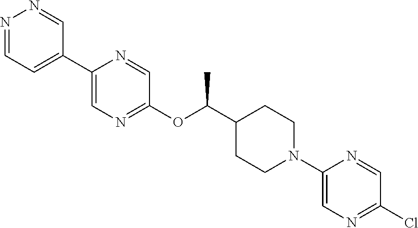

A mixture of 4-(5-{[(1S)-1-(4-piperidinypethyl]oxy}-2-pyrazinyl)pyridazine trifluoroacetate (prepared as in Example 3, Step 1, 50 mg, 0.125 mmol), 2,5-dichloropyrazine (19 mg, 0.13 mmol) and potassium carbonate (52 mg, 0.38 mmol) in DMSO (1 mL) was stirred at 100° C. overnight. The mixture was filtered, and the resulting filtrate was purified by reverse-phase preparative HPLC using CH₃CN:H₂O gradient (10:90 to 100:0) with 0.2% NH₄OH as a modifier to give the title compound (25 mg, 42%) as a tan solid. ¹H NMR (400 MHz, CDCl₃): δ 9.73 (s, H), 9.24 (d, 1H, J=5.4 Hz), 8.61 (d, 1H, J=1.3 Hz), 8.28 (d, 1H, J=1.3 Hz), 8.01 (d, 1H), 7.96 (dd, 1H, J=5.4, 2.4 Hz), 7.84 (d, 1H, J=1.3 Hz), 5.16 (m, 1H), 4.31 (t, 2H, J=11.5 Hz), 2.87 (dt, 2H, J=12.9, 2.8 Hz), 1.98-1.78 (m, 3H), 1.51-1.30 (m, 5H); LRMS (ESI), m/z 398 (M+H).

### Example 8

### 1,1-Dimethylethyl 4-((1S)-1-{[5-(5-methyl-4-pyridazinyl)-2-pyrazinyl]oxy}ethyl)-1-piperidinecarboxylate

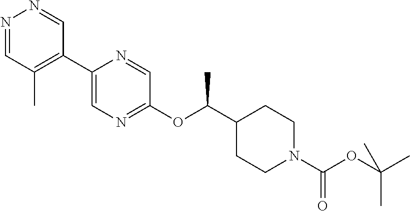

A mixture of 4-methyl-5-(tributylstannanyl)pyridazine (1.0 g, 2.64 mmol), 1,1-dimethylethyl 4-{(1S)-1-[(5-bromo-2-pyrazinyl)oxy]ethyl}-1-piperidinecarboxylate (prepared as in Example 1, Step 5, 600 mg, 1.55 mmol), bis(triphenylphosphine)palladium(II) chloride (109 mg, 0.155 mmol) and copper(I) iodide (59 mg, 0.31 mmol) in DMF (7 mL) was degassed with nitrogen for 15 min. The mixture was stirred in a microwave at 140° C. for 30 min, cooled to room temperature, poured onto a 10% KF (aq) solution, then diluted with EtOAc, and stirred at room temperature for 1 h. The mixture was extracted with EtOAc. The organics were pooled, filtered through a ChemElut (10 mL) column, and eluted with EtOAc. The organics were concentrated to dryness and the resulting residue was purified by chromatography on an amino-silica gel column using 0 to 30% EtOAc/hexanes gradient to give the title compound (609 mg, 98%) as an orange solid. ¹H NMR (400 MHz, CDCl₃): δ 9.21 (s, 1H), 9.09 (s, 1H), 8.29 (d, 2H, J=3.4 Hz), 5.12 (m, 1H), 4.15 (bs, 2H), 2.75-2.62 (m, 2H), 2.51 (s, 3H), 1.86-1.64 (m, 3H), 1.43 (s, 9H), 1.35-1.25 (m, 5H); LRMS (ESI), m/z 400 (M+H).

### Example 9

### 4-Methylphenyl 4-((1S)-1-{[5-(4-pyridazinyl)-2-pyrazinyl]oxy}ethyl)-1-piperidinecarboxylate

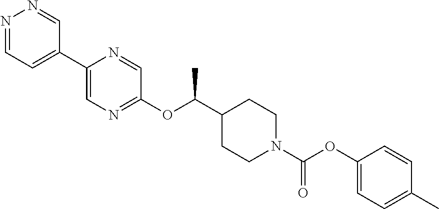

4-Methylphenyl chloridocarbonate (0.018 mL, 0.125 mmol) was added dropwise to a solution of 4-(5-{[(1S)-1-(4-piperidinypethyl]oxy}-2-pyrazinyl)pyridazine trifluoroacetate (prepared as in Example 3, Step 1, 50 mg, 0.125 mmol), Hunig's base (0.109 mL, 0.626 mmol), and DCM (3 mL) at room temperature. The mixture was stirred at room temperature overnight and then the mixture was concentrated to dryness. The resulting residue was purified by reverse-phase preparative HPLC using CH₃CN:H₂O gradient (10:90 to 100:0) with 0.2% NH₄OH as a modifier to give the title compound (34 mg, 65%) as a white solid. ¹H NMR (400 MHz, CDCl₃): δ 9.75 (d, 1H, J=1.3 Hz), 9.26 (d, 1H, J=4.9 Hz), 8.64 (s, 1H), 8.30 (s, 1H), 8.02 (dd, 1H, J=5.4, 2.4 Hz), 7.12 (d, 2H, J=8.2 Hz), 6.95 (d, 2H, J=8.4 Hz), 5.19 (m, 1H), 4.34 (bs, 2H), 3.03-2.73 (m, 2H), 2.30 (s, 3H), 1.94-1.74 (m, 3H), 1.51-1.31 (m, 5H); LRMS (ESI), m/z 420 (M+H).

### Example 10

### 4-{5-[((1S)-1-{1-[5-(1-Methylethyl)-2-pyrimidinyl]-4-piperidinyl}ethyl)oxy]-2-pyrazinyl}pyridazine

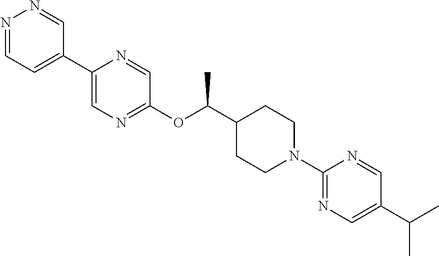

A mixture of 4-(5-{[(1S)-1-(4-piperidinypethyl]oxy}-2-pyrazinyl)pyridazine trifluoroacetate (prepared as in Example 3, Step 1, 100 mg, 0.250 mmol), 2-chloro-5-(1-methylethyl)pyrimidine (47 mg, 0.30 mmol) and potassium carbonate (104 mg, 0.75 mmol) in DMSO (1 mL) was stirred at 100° C. for 3 h. The mixture was filtered, and the resulting filtrate concentrated to dryness. The resulting residue was purified by reverse-phase preparative HPLC using CH₃CN:H₂O gradient with 10 mM NH₄OH as a modifier to give the title compound (55 mg, 54%) as an off-white solid. ¹H NMR (400 MHz, DMSO-d₆): δ 9.87 (s, 1H), 9.34 (d, 1H, J=5.5 Hz), 9.08 (s, 1H), 8.47 (s, 1H), 8.27 (s, 2H), 8.24 (dd, 1H, J=5.5, 2.3 Hz), 5.16 (quin, 1H, J=6.2 Hz), 4.72 (d, 2H, J=11.3 Hz), 2.89-2.70 (m, 3H), 2.03-1.91 (m, 1H), 1.87 (d, 1H, J=12.9 Hz), 1.76 (d, 1H, J=12.7 Hz), 1.36-1.16 (m, 11H); LRMS (ESI), m/z 406 (M+H).

### Example A

### Tests for Agonists of GPR119

The assay consists of CHO-K1 6CRE-luciferase cells that stably express human GPR119 receptor plated at 15000 cells/well in Dulbecco's Modified Eagle Medium: Nutrient Mixture F-12 (DMEM/F12), 5% Fetal Bovine Serum (FBS), 2 mM I-glutamine in black 384-well assay plates. On the following day, the media is removed by aspiration and replaced with 20 L of DMEM/F12, 2 mM I-glutamine (no FBS) utilizing a Matrix Multidrop. Test compounds (25 μL) are then pipetted into the assay plate using a Packard Minitrak. The plates are then incubated for 5 hours at 37° C. Under subdued light conditions, 15 μL of a 1:1 solution containing SteadyLite™ and Dulbecco's Phosphate Buffered Saline with 1 mM CaCl₂ and 1 mM MgCl₂ is added to the plates using a Matrix Multidrop. Plates are then sealed with self-adhesive clear plate seals and the amount of luciferase generated is quantified in a Wallac Viewlux™. Compounds are also tested in the same manner against cells without the GPR119 receptor so as to check for false positives.

GPR119 agonists will generally show an increase, in a concentration dependent manner, in intracellular calcium levels and, generally, have an EC₅₀<10 μM.

1. A compound of the formula (I) or a pharmaceutically acceptable salt thereof: 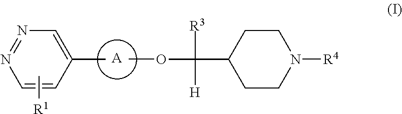
wherein
R¹ is selected from the group consisting of —C₁₋₃alkyl or halogen;
ring A is selected from the group consisting of: 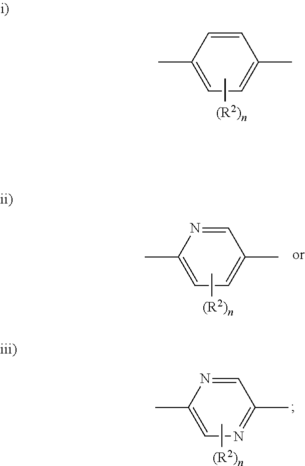
R² is a replacement for a hydrogen atom and is independently selected from the group consisting of halogen, —CF₃, —OH, —C₃₋₇cycloalkyl, and —C₁₋₅alkoxyl;
n is 0, 1, or 2;
R³ is selected from a group consisting of —H, —C₁₋₅alkyl, or —C₃₋₇cycloalkyl;
R⁴ is —C(O)C(O)R⁵, —C(O)OR⁵, —C(O)R⁵, —S(O)₂C₁₋₅alkyl, —S(O)₂C₃₋₇cycloalkyl, —S(O)₂NR⁶R⁷, Ar, —CH₂Ar, —C(O)NHC₁₋₅alkyl, —C(O)NHC₃₋₇cycloalkyl, —C(O)NHC₁₋₅alkyl-Ar, or —C(O)NR⁶R⁷;
R⁵ is independently selected from the group consisting of
—C₁₋₅alkyl,
—C₃₋₇cycloalkyl,
phenyl,
phenyl(C₁₋₄alkylene),
a heterocyclic group of 3-7 ring members, and
—C₁₋₅alkyl substituted by a heterocyclic group of 3-7 ring members,
which group members may be further optionally substituted by one or more of halogen, C₁₋₅alkoxyl, a heteroaryl ring of 5-6 members,
—NR⁶R⁷, or —C(O)NR⁶R⁷;
R⁶ and R⁷ are independently selected from the group consisting of —H, —C₃₋₇cycloalkyl, and a heterocyclic group of 3-7 members or R⁶ and R⁷ are alkyl and together combine to form a ring having 4 to 7 ring atoms and optionally containing a heterogroup selected from —O—, —NH—, and —N(C₁₋₅alkyl)- and wherein said ring having 4 to 7 ring atoms is optionally substituted by oxo; and
Ar is aryl or a 5- or 6-membered heteroaryl group, which may be substituted by one or more substituents independently selected from halogen, —CF₃, —C₁₋₅alkyl, —C₃₋₇cycloalkyl, —CN, —NR⁶R⁷, and —NO₂.

2. The compound according to claim 1, wherein R¹ is —CH₃.

3. The compound according to claim 1, wherein ring A is of formula i), ii) or iii), with n=0.

4. The compound according to claim 1, wherein R² is selected from the group consisting of —H, —F, —CH₃, —OCH₃.

5. The compound according to claim 1, wherein R³ is —CH₃.

6. A compound according to claim 5 wherein R³ is —CH₃ and the stereochemistry of the stereogenic carbon is (S).

7. The compound according to claim 1, wherein R⁴ is —C(O)OCH(CH₃)₂.

8. A compound according to claim 1 wherein ring A is:

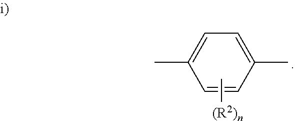

9. A compound according to claim 1 wherein ring A is:

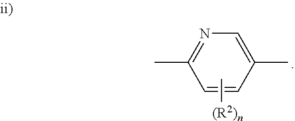

10. A compound according to claim 1 wherein ring A is:

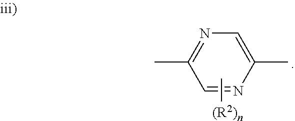

11. A compound according to claim 1 wherein R⁴ is Ar as shown below: 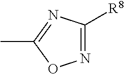
in which R⁸ is selected from a group consisting of
—C₁₋₅alkyl and —C₃₋₇cycloalkyl.

12. A compound selected from
1-Methylethyl 4-((1S)-1-{[5-(4-pyridazinyl)-2-pyrazinyl]oxy}ethyl)-1-piperidinecarboxylate
4-{5-[((1S)-1-{1-[3-(1-Methylethyl)-1,2,4-oxadiazol-5-yl]-4-piperidinyl}ethyl)oxy]-2-pyrazinyl}pyridazine
2-Methylpropyl 4-((1S)-1-{[5-(4-pyridazinyl)-2-pyrazinyl]oxy}ethyl)-1-piperidinecarboxylate
Phenyl 4-((1S)-1-{[5-(4-pyridazinyl)-2-pyrazinyl]oxy}ethyl)-1-piperidinecarboxylate 4-[5-({(1S)-1-[1-(5-Ethyl-2-pyrimidinyl)-4-piperidinyl]ethyl}oxy)-2-pyrazinyl]pyridazine
4-[5-({(1S)-1-[1-(5-Methyl-2-pyrimidinyl)-4-piperidinyl]ethyl}oxy)-2-pyrazinyl]pyridazine
4-[5-({(1S)-1-[1-(5-Chloro-2-pyrazinyl)-4-piperidinyl]ethyl}oxy)-2-pyrazinyl]pyridazine
1,1-Dimethylethyl 4-((1S)-1-{[5-(5-methyl-4-pyridazinyl)-2-pyrazinyl]oxy}ethyl)-1-piperidinecarboxylate
4-Methylphenyl 4-((1S)-1-{([5-(4-pyridazinyl)-2-pyrazinyl]oxy}ethyl)-1-piperidinecarboxylate
4-{5-[((1S)-1-{1-[5-(1-Methylethyl)-2-pyrimidinyl]-4-piperidinyl}ethyl)oxy]-2-pyrazinyl}pyridazine
and pharmaceutically acceptable salts thereof.

13.-20. (canceled)

21. A method for the treatment of metabolic disorders or conditions comprising the administration of a compound according to claim 1.

22. The method of claim 21 wherein the metabolic disorder is selected from the group consisting of diabetes and obesity.

23. A pharmaceutical composition comprising a compound of claim 1 or a salt thereof and at least one pharmaceutically acceptable carrier, diluent, or excipient.

24. A process for the preparation of a pharmaceutical composition comprising admixing a compound of claim 1 or a salt thereof with at least one pharmaceutically acceptable carrier, diluent, or excipient.

25. A compound according to claim 1 or a salt thereof in combination with at least one therapeutic agent.
